(19) United States
(12) Patent Application Publication
(10) Pub. No.: US 2017/0157219 A1
(43) Pub. Date: Jun. 8, 2017

(54) METHODS OF TREATING ZIKA VIRUS, MERS-COV, CHIKUNGUNYA, VENEZUELAN EQUINE ENCEPHALITUS, AND RHINOVIRUS IN MAMMALIAN PATIENTS

(71) Applicant: Tamir Biotechnology, Inc., San Diego, CA (US)

(72) Inventor: Thomas W. Hodge, III, Athens, GA (US)

(73) Assignee: Tamir Biotechnology, Inc., San Diego, CA (US)

(21) Appl. No.: 15/429,046

(22) Filed: Feb. 9, 2017

Related U.S. Application Data
(63) Continuation-in-part of application No. 14/736,170, filed on Jun. 10, 2015.
Application No. 14/736,170 is a continuation-in-part of application No. 14/667,282, filed on Mar. 24, 2015, now abandoned.
Application No. 14/667,282 is a continuation-in-part of application No. 14/316,893, filed on Jun. 27, 2014, now abandoned.
Application No. 14/316,893 is a continuation-in-part of application No. 14/247,723, filed on Apr. 8, 2014, now abandoned.
Application No. 14/247,723 is a continuation-in-part of application No. 14/229,816, filed on Mar. 28, 2014, now abandoned.
(60) Provisional application No. 62/295,842, filed on Feb. 16, 2016.
(60) Provisional application No. 62/147,209, filed on Apr. 14, 2015.
(60) Provisional application No. 62/102,671, filed on Jan. 13, 2015.
(60) Provisional application No. 62/063,551, filed on Oct. 14, 2014.
(60) Provisional application No. 62/040,885, filed on Aug. 22, 2014.

(51) Int. Cl. A61K 38/46

(52) U.S. Cl. CPC A61K 38/465; C12Y 301/27

(57) ABSTRACT

Viral infections in mammals can be treated and prophylactically prevented by systemic administration of ranpirnase and three other ribonucleases that are highly homologous with it and that have activities that are highly similar to it. Experimental results against Zika virus, Middle East Respiratory Syndrome Coronavirus (“MERS-CoV”), Chikungunya virus, Venezuelan equine encephalitis, and rhinovirus-14 are disclosed.

### BACKGROUND OF THE INVENTION

The invention relates to treatment of infections caused by viruses (other than Dengue fever and yellow fever) classified in Baltimore Group IV, and more particularly relates to treatment of such infections in mammalian patients and especially such infections in humans. In its most immediate sense, the invention relates to treatment of infections caused by viruses in the Flaviviridae family (all of which are classified in Baltimore Group IV), and specifically infections caused by the Zika virus.

Ranpirnase has previously demonstrated activity against a large number of viral infections, some of which are members of the Flaviviridae family (which virus family is classified in Baltimore Group IV). Recent experiments have now shown that ranpirnase is active against Zika virus in Huh-7 human liver carcinoma cells. This experimental evidence provides additional justification for the conclusion that ranpirnase is active against all viruses classified in Baltimore Group IV.

### SUMMARY OF THE INVENTION

Recent experiments have shown that ranpirnase demonstrates surprisingly strong antiviral effects against a surprisingly large number of different viruses, including viruses (e.g. MERS-CoV and EBOV) that are highly resistant to treatment.

It is believed that the surprisingly broad-spectrum activity of the invention comes from the ways in which ranpirnase degrades various forms of RNA. To date, three RNA-degrading mechanisms appear to be relevant to antiviral therapy using ranpirnase.

The first of these mechanisms is degradation of tRNA. Degrading tRNA inside a mammalian cell makes that cell resistant to some viral infections. This is because some viruses replicate by protein synthesis using the ribosome, and protein synthesis cannot occur unless transfer RNAs enter the ribosome to deliver the amino acids needed to synthesize the protein. Thus, a systemic application of an agent that degrades tRNA will prevent or at least substantially impede some viruses from spreading to uninfected cells. If this application occurs before the virus has spread widely enough to endanger the host mammal, the virus will eventually die.

The second mechanism is degradation of viral double-stranded RNA. Some viruses produce double-stranded RNA as part of their process of proliferation in mammalian cells, and destroying that double-stranded RNA can prevent or at least substantially impede replication of such viruses.

The third mechanism is degradation of microRNA and siRNA. In certain viruses that proliferate using double-stranded RNA, that double-stranded RNA is produced by the interaction of microRNA or siRNA with single-stranded RNA. Destroying the microRNA or siRNA can prevent the formation of the viral double-stranded RNA by which the virus replicates.

Ranpirnase is known to degrade each of these RNAs. It degrades tRNA very effectively (see Lin et al., Biochemical and Biophysical Research Communications 201 (1), 156-162 (1994)). And, because normal mammalian cells degrade approximately 80% of their tRNA as a natural process, this degradation causes little if any harm to the cells themselves. As a result, except in instances where a viral infection has spread too far to be effectively controlled, a systemic application of ranpirnase causes some viruses to die out without killing the normal cells that those viruses infect.

Ranpirnase is known to degrade double-stranded RNA as well (see Saxena et al., Anticancer Res. 29 (4), 1067-1071 (April 2009)). And, ranpirnase is known to degrade certain microRNA (see Goparaju et al., Oncogene 30 (24), 2767-2777, (Jun. 16, 2011)) and certain siRNA (see Zhao et al., Cell Cycle. 7 (20), 3258-3261 (October 2008)).

Hence, it appears that the RNA-degrading characteristics of ranpirnase make it possible to use ranpirnase as a pharmaceutical for treating a wide variety of viral infections in mammals by administering ranpirnase systemically. And, it also appears that these characteristics permit ranpirnase to be administered prophylactically as well as therapeutically.

Furthermore, it is believed that other ribonucleases will have the same antiviral effects that ranpirnase has. These other ribonucleases (including the below-identified “'805 variant”, “Amphinase 2”, and rAmphinase 2”) are highly homologous to ranpirnase and have exhibited antiviral properties that are highly similar to those of ranpirnase when treating other viral infections.

### BRIEF DESCRIPTION OF THE DRAWINGS

In the drawings, wherein

CC₅₀ is the cytotoxic concentration (expressed in nM) of ranpirnase, i.e. the ranpirnase concentration that decreased cell viability by 50%, and

IC₅₀ is the inhibitory concentration (expressed in nM) of ranpirnase, i.e. the ranpirnase concentration that inhibited replication of the virus under test by 50%,

SI, the selective index, is CC_{50/}IC₅₀. The higher the value of SI, the more active is the ranpirnase against the virus under test.

FIG. 1 shows the results of testing the anti-viral activity of ranpirnase against Zika virus in Huh-7 human liver carcinoma cells;

FIG. 2 shows the results of testing the anti-viral activity of various concentrations of ranpirnase against MERS-CoV virus in normal human bronchial epithelial (“NHBE”) cells, compared with the anti-viral activities of various concentrations of SARS protease inhibitor and Infergen;

FIG. 3 shows the AC50 toxicity values for ranpirnase inhibition of VEEV and CHIV;

FIG. 4 shows results of quality control (QC) testing of ranpirnase against VEEV infection in astroctyes;

FIG. 5 shows the effect of ranpirnase against VEEV infection in astrocytes.

FIG. 6 shows results of QC testing of ranpirnase against VEEV infection in HeLa cells;

FIG. 7 shows the effect of ranpirnase against VEEV infection in HeLa cells;

FIG. 8 shows results of QC testing of ranpirnase against CHIV infection in U2OS cells;

FIG. 9 shows the effect of ranpirnase against CHIV infection in U2OS cells;

FIG. 10 shows the doses of ranpirnase used in a study of ranpirnase inhibition of RV-14 in NHBE cells; and

FIG. 11 shows the results of the study of FIG. 10.

### DETAILED DESCRIPTION OF PREFERRED EMBODIMENTS

### Example 1

### Zika Virus in Huh-7 Liver Carcinoma Cells

The antiviral activity of ranpirnase against Zika virus strain Uganda MR 766 in Huh-7 human liver carcinoma cells was assessed. Interferon (which is known to be active against this Zika virus strain) was run in parallel as a control.

The ranpirnase and the control were serially diluted to produce eight half-log dilutions in MEM medium. The diluent for ranpirnase was 50 μg/mL gentamicin and serum; the diluent for interferon was 50 μg/mL gentamicin and serum and trypsin. Each dilution was added to 5 wells of a 96-well plate with 80%-100% confluent cells, and three wells of each dilution were then infected. Two wells remained uninfected as toxicity controls.

The virus was incubated for 4 days at 37° C. and 5% CO₂. After cytopathic effect (CPE) was observed microscopically, plates were scored for degree of CPE and then stained with neutral red dye for approximately 2 hours, then supernatant dye was washed from the wells and the incorporated dye was extracted in 50:50 Sorensen citrate buffer/ethanol and read on a spectrophotometer. The optical density of test wells was converted to percent of cell and virus controls, then the concentration of ranpirnase required to inhibit CPE by 50% (IC₅₀) was calculated by regression analysis. The concentration of ranpirnase that would cause 50% CPE in the absence of virus (CC₅₀) was similarly calculated, as was the selectivity index SI.

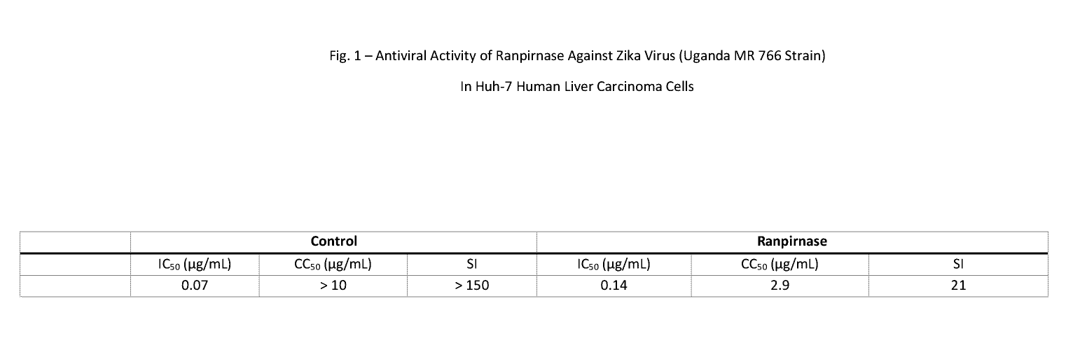

The results of this experiment are shown in FIG. 1. Ranpirnase demonstrated a selectivity index of 21, indicating activity against the Uganda MR 766 strain of Zika virus.

The selectivity index SI is an accepted measurement of the ability of a drug under test to inhibit replication of a viral infection without killing the infected cells. Where SI in the accompanying Figure is greater than 1, ranpirnase is active against the virus indicated, and increasing values of SI indicate increasing activity. Thus, as can be seen in FIG. 1, ranpirnase is active against the Uganda MR766 strain of Zika virus in the Huh-7 human liver carcinoma.

Because SI measures the ability of a substance under test to inhibit replication of a particular virus without killing the infected cells themselves, it is reasonably correlated with usefulness of the substance in treating a mammalian subject that is infected with the virus. Accordingly, test results in which SI>1 indicate that mammalian subjects infected with with Uganda MR 766 strain of Zika virus can be treated by systemic administration of an appropriate dose of ranpirnase. Additionally, activity against the Uganda MR 766 strain of the Zika virus is reasonably correlated with activity against all strains of the Zika virus because such strains are similar and behave similarly. Furthermore, other below-disclosed experimental results in VEEV, CHIV, and RV-14 indicate that it should be possible to use ranpirnase as a prophylactic to prevent Zika viral infection.

### Example 2

### MERS-CoV in NHBE Cells

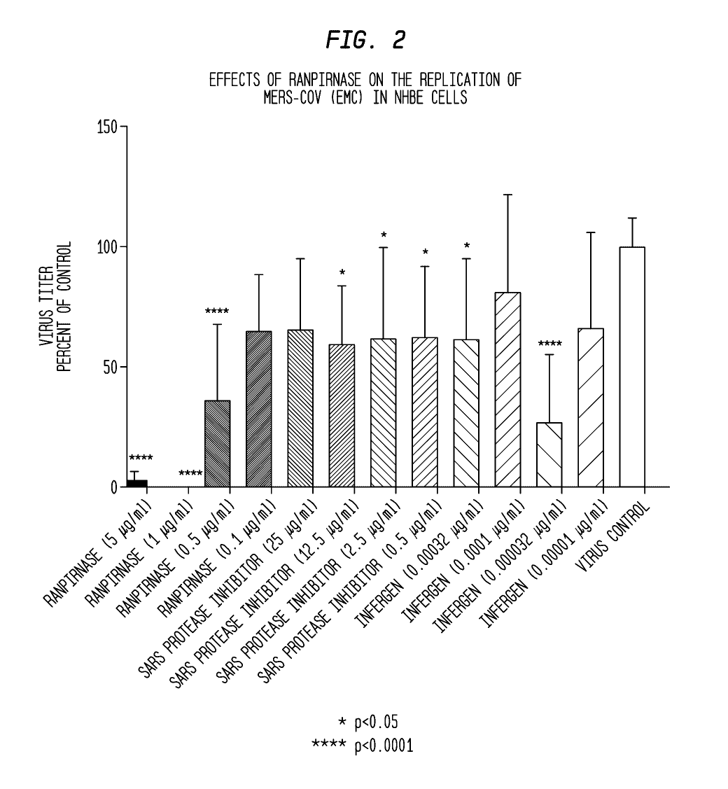

In the experiment illustrated in FIG. 2, the anti-viral activity of ranpirnase against MERS-CoV virus was compared to the activities of two known anti-viral agents: SARS protease inhibitor and Infergen. The experiment was carried out using four different concentrations of each agent on normal human bronchial epithelial (NHBE) cells.

More specifically, the NHBE cells were grown in HEPES Buffered Saline Solution at 37° C. for seven days. The cells were washed and refreshed once daily. Two controls were used: one contained MERS-CoV virus and the other contained uninfected NHBE cells that were treated with the agents under test.

On the eighth day, the tested concentrations of the three agents under test were introduced into the cells and buffer solution and the virus was introduced at a multiplicity of infection (“MOI”) of 0.01. The virus- and agent-containing samples were then incubated for 72 hours at 37° C. and 5% CO₂, with the medium being replenished once each day. After 72 hours, the samples were then titrated to determine their viral content.

In this experiment SI was not calculated. Rather, the anti-viral activity of the various agents under test was determined by comparing viral production (viral titer in Vero 76 cells) in NHBE cells that had been treated with the various agents under test to the viral production in the NHBE cells used as controls.

As can be seen in FIG. 2, ranpirnase was far more active against MERS-CoV virus than either of the other agents. And, the activity of ranpirnase was clearly statistically significant, since all but the least concentrated of the doses of ranpirnase had a p value of less than 0.0001. This reduction of viral titers of MERS-CoV virus without killing the host cells is reasonably correlated with usefulness of ranpirnase in treating MERS-CoV.

Because ranpirnase was so effective at inhibiting replication of the MERS-CoV virus in NHBE cells while not killing the host cells, this experiment further evidences the likelihood that systemically administered ranpirnase will be useful in treating a mammalian subject infected with a virus, and particularly a mammalian subject infected with MERS-CoV virus. Furthermore, other below-disclosed experimental results in VEEV, CHIV, and EBOV indicate that it should be possible to use ranpirnase as a prophylactic to prevent MERS-CoV infection.

### Example 3

### VEEV, and CHIV (In Vitro)

Methodology

Several studies were conducted to assess the ability of ranpirnase to inhibit infection of cells by VEEV and CHIV. Ranpirnase solution and powder-derived ranpirnase were tested. The powder-derived ranpirnase was lyophilized ranpirnase provided by Tamir Biotechnology, Inc. Quality control of the assay was conducted using Positive (Neutral) control (n=16) or infected cells+media, uninfected cells (Negative control) (n=16) and dose response for control inhibitors (n=2 or 4). Z′ was calculated for Neutral control and uninfected cells. Data were normalized on the plate bases. Data analysis was done using GeneData software and analysis of dose response curve to determine ED50 of ranpirnase was performed using GeneDataCondoseo software applying Levenberg-Marquardt algorithm (LMA) for curve fitting strategy.

VEEV in Astrocytes

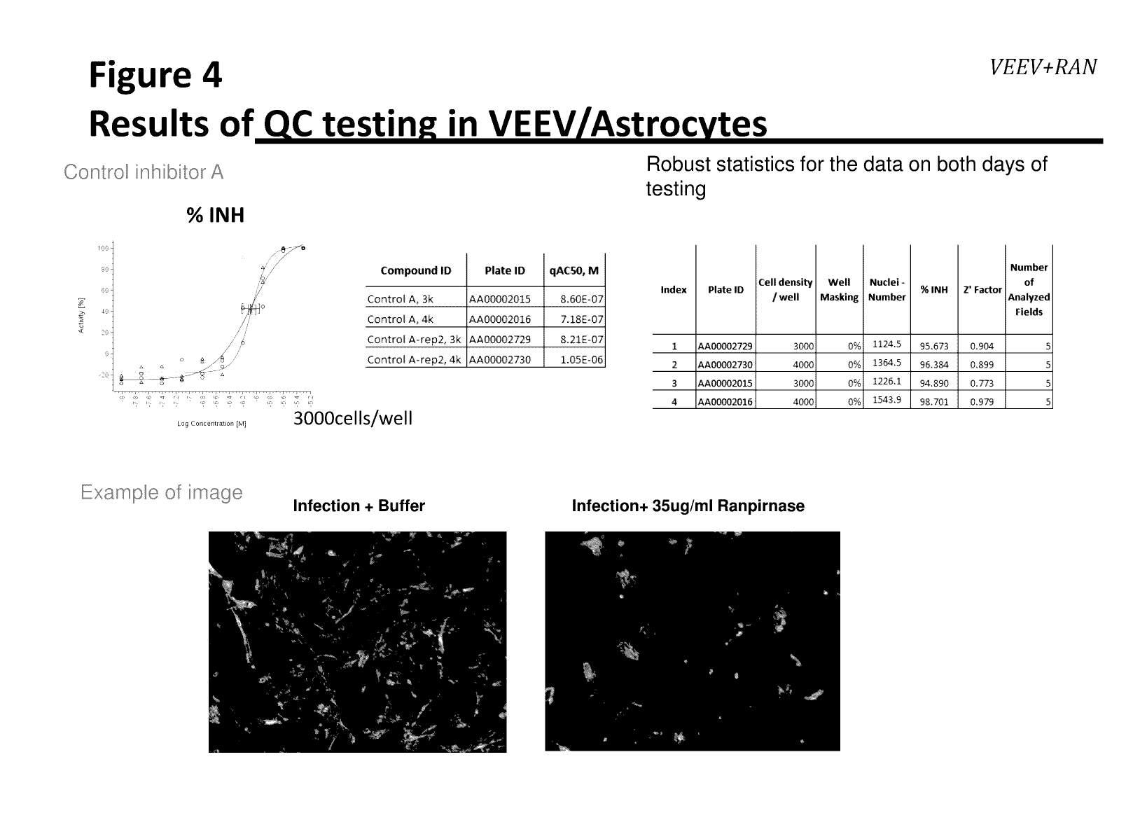

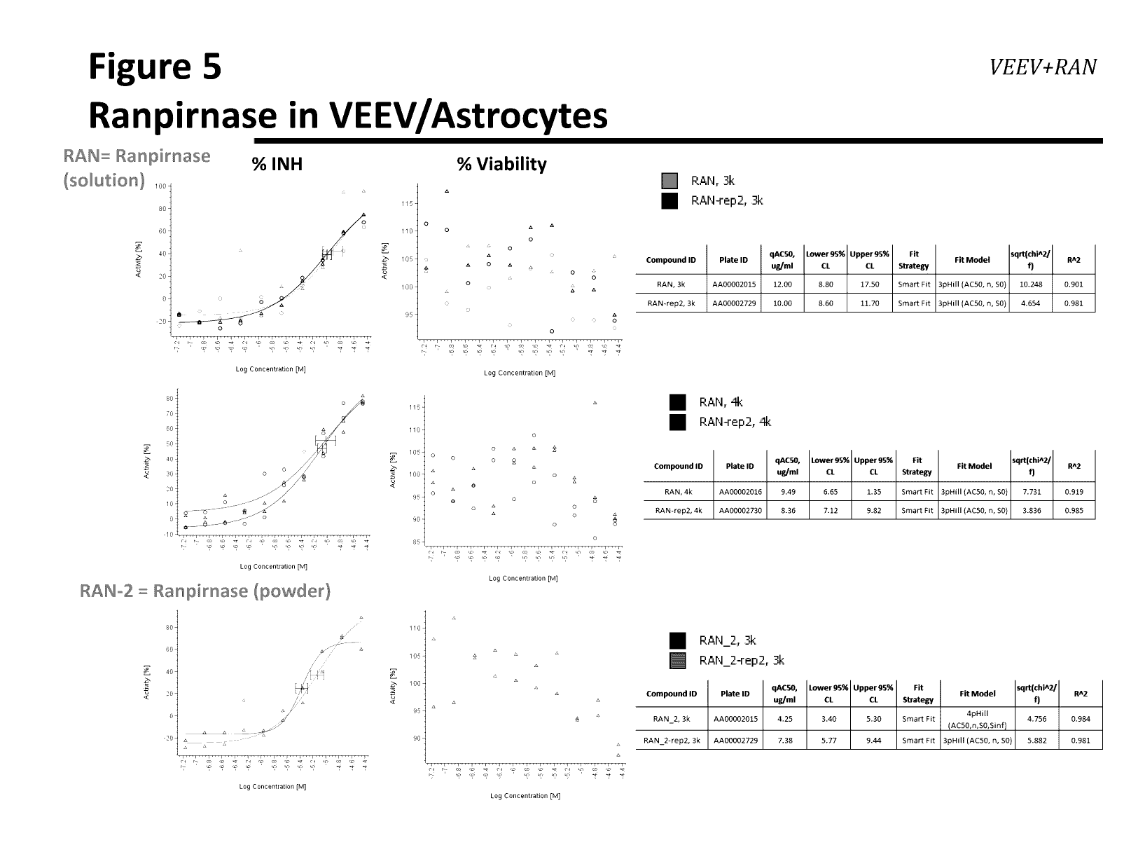

To test the effect of ranpirnase on VEEV infection of astrocytes, ranpirnase solution (“RAN”) was tested in duplicated 10 point dose response, and powder-derived ranpirnase (“RAN-2”) was re-suspended in phosphate buffered saline at 3.5 mg/ml and was tested only as a single dose response. Both the RAN and the RAN-2 were tested in two independent experiments. In these experiments, astrocytes were plated at 4,000 and 3,000 cells/well, incubated overnight and pre-treated with ranpirnase for 2 hours before the infection. Cells were infected at a multiplicity of infection (“MOI”) equal to 0.05 for 20 hours. The results of the study are provided in FIGS. 4 and 5.

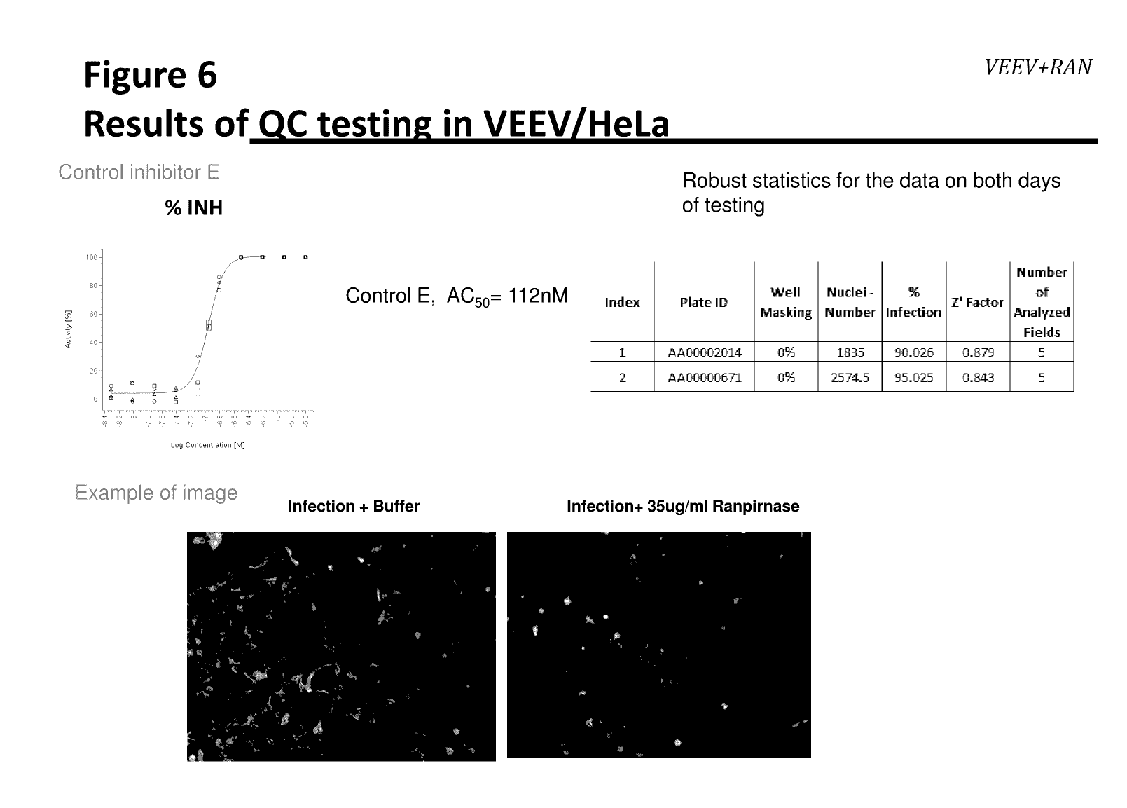

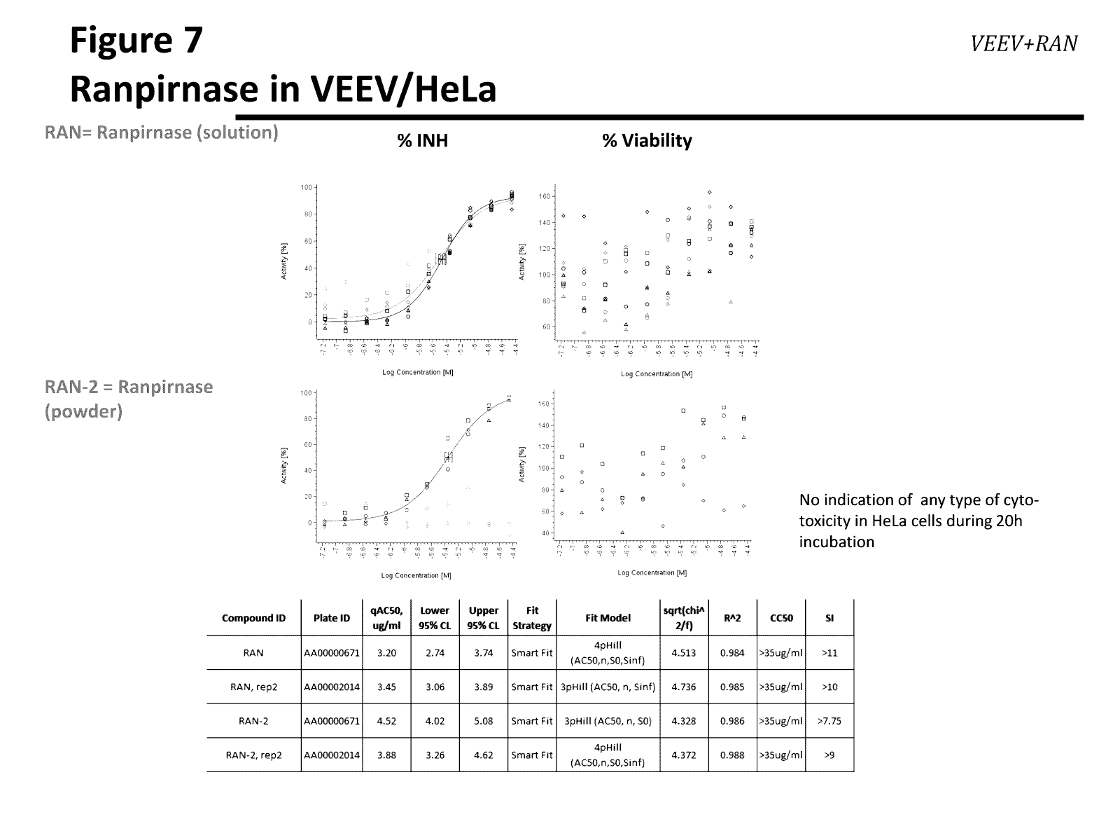

To test the effect of ranpirnase on VEEV infection of HeLa cells, RAN was tested in quadruplicated (n=4) 10 point dose response repeated in two independent experiments (rep1 and rep2). RAN2 was tested in n=2 dose responses on plate and repeated in 2 independent experiments. HeLa cells were plated at 4,000 cells/well, incubated overnight and pre-treated with ranpirnase 2 hours before infection. Cells were infected at an MOI equal to 0.05 for 20 hours. The results of the study are provided in FIGS. 6 and 7. As shown in FIG. 7, SI values were over 10 for RAN and over 7.75 for RAN-2.

CHIV in U2OS Cells

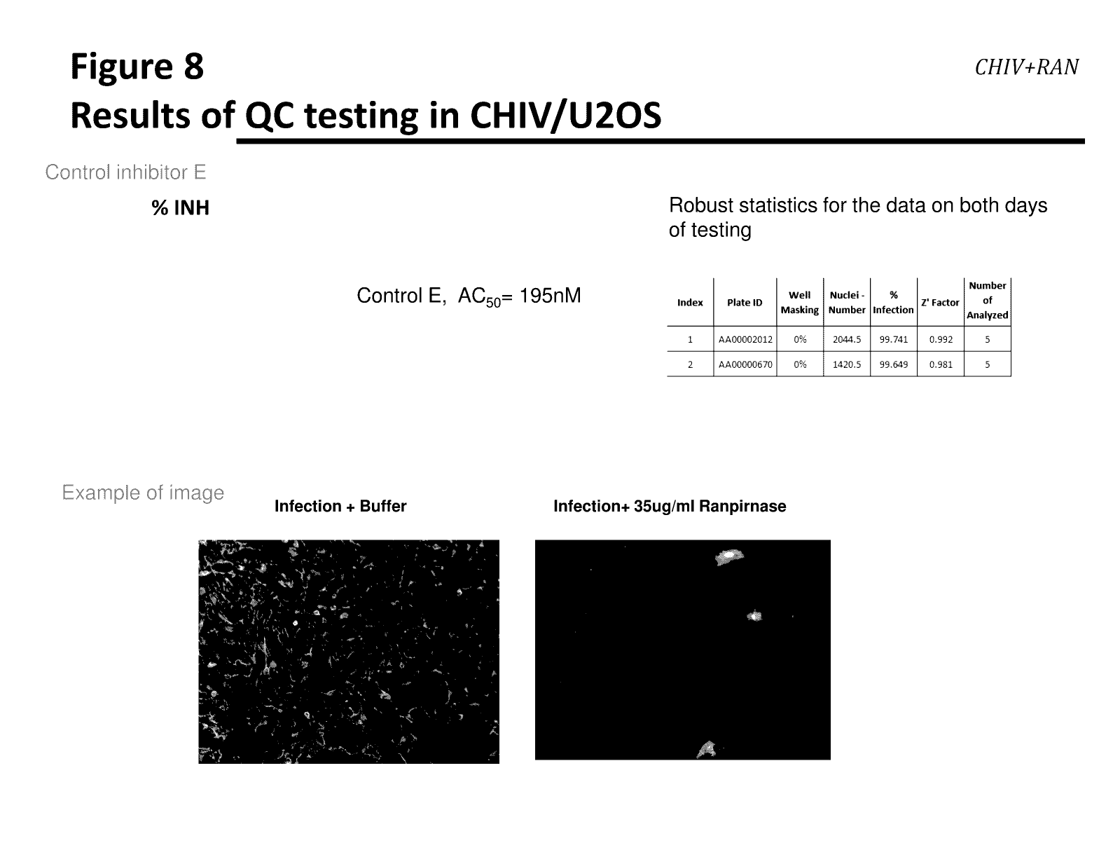

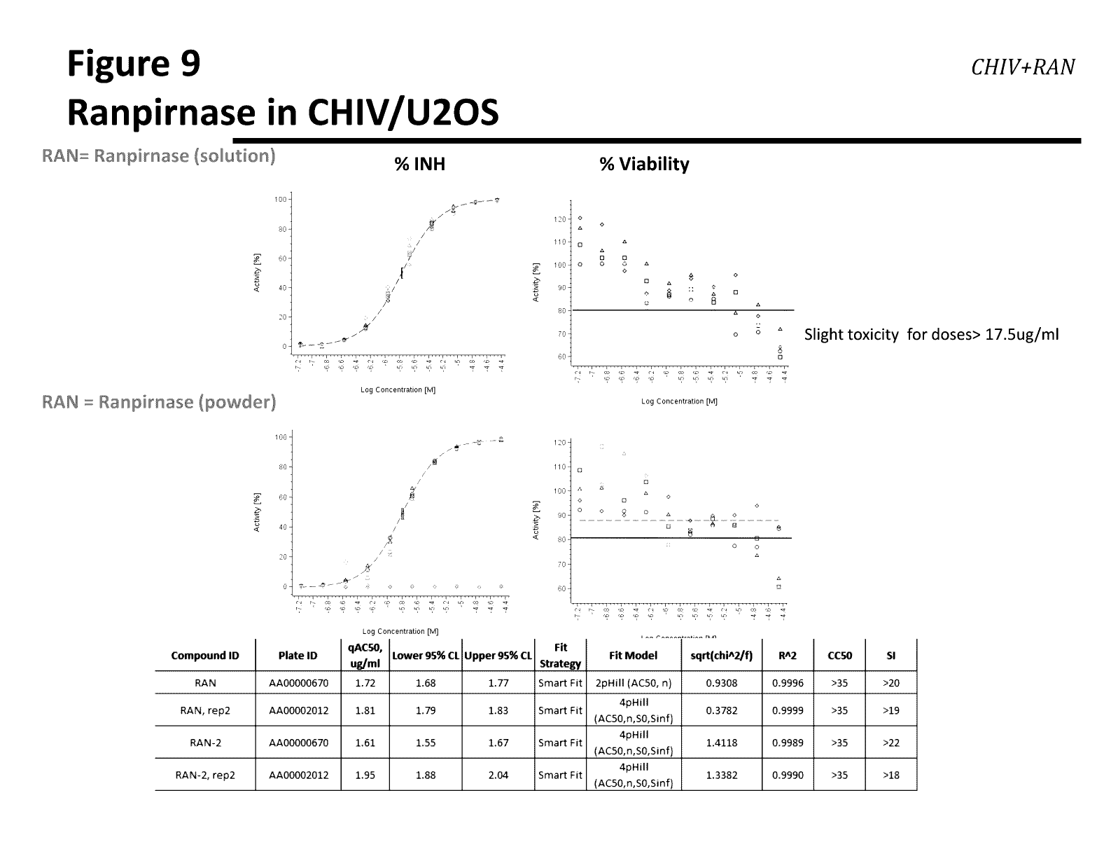

To test the effect of ranpirnase on CHIV infection of U2OS cells, ranpirnase solution was tested in quadruplicated (n=4) 10 point dose response repeated in two independent experiments (rep1 and rep2). The RAN2 stock was tested in n=2 dose responses on plate and repeated in two independent experiments. U2OS cells were plated at 3,000 cells/well, incubated overnight and pre-treated with ranpirnase 2 hours before infection. Cells were infected at a MOI equal to 0.4 for 24 hours. The results of the study are provided in FIGS. 8 and 9. As shown in FIG. 9, SI values were over 18.

Summary of In Vitro VEEV and CHIV Experiments

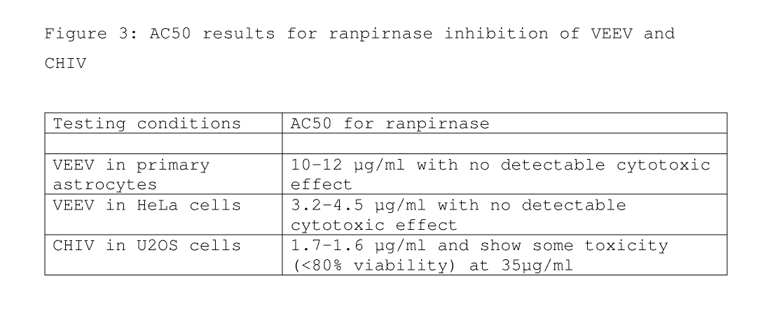

The results of the study showed that ranpirnase exhibited robust inhibition of VEEV and CHIV, with surprisingly low AC50 values and surprisingly high SI values. Because SI measures the ability of a substance under test to inhibit replication of a particular virus without killing the infected cells themselves, it is reasonably correlated with usefulness of the substance in treating a mammalian subject that is infected with the virus. Accordingly, test results such as these in which SI>1 indicate that mammalian subjects infected with VEEV, and CHIV can be treated by systemic administration of an appropriate dose of ranpirnase. FIG. 3 provides an overall summary of the AC50 results of the studies. AC50 indicates the concentration of the tested agent—here, ranpirnase—that produce half the maximum inhibition of the virus being inhibited.

These experiments demonstrate that ranpirnase inhibited replication of the tested VEEV and CHIV in various mammalian cells (astrocytes, U2OS cells) without killing the cells themselves. These experiments further evidence the likelihood that systemically administered ranpirnase will be useful in treating a mammalian subject infected with a virus, and particularly a mammalian subject infected with VEEV and CHIV. Furthermore, it is to be noted that in these experiments, the ranpirnase was used prophylactically, in that the viruses were introduced into cells that had already been treated with ranpirnase. These experiments therefore constitute evidence that the antiviral qualities of ranpirnase can be used prophylactically as well as therapeutically.

### Example 4

### RV in NHBE Cells

Ranpirnase stock solution was prepared, stored, thawed, and used to prepare working solutions as described in the AV and RSV experiments disclosed above.

NHBE from MatTek Corporation were used in the study. They were the same cell line as were used in the AV and RSV experiments discussed above and were provided in the same kits. As in the AV and RSV experiments, tissue inserts were immediately transferred to individual wells of a 6-well plate according to manufacturer's instructions. Tissues were supplied with 1 ml of the same culture medium used in the AV and RSV experiments to the basolateral side, and the apical side was exposed to a humidified 95% air/5% CO₂ environment. Cells were equilibrated as in the AV experiment, and after this equilibration period, the mucin layer was removed as in the AV and RSV experiments and the culture medium was replenished.

RV-14 (strain 1059 from ATCC) was stored at −80° C. prior to use. The titer of the stock virus was equal to titer 3.6 log 10 CCID50/0.1 ml. The dose level of challenge virus was based on data from the previous experiments, and corresponded to a multiplicity of infection (MOI) of 0.0041.

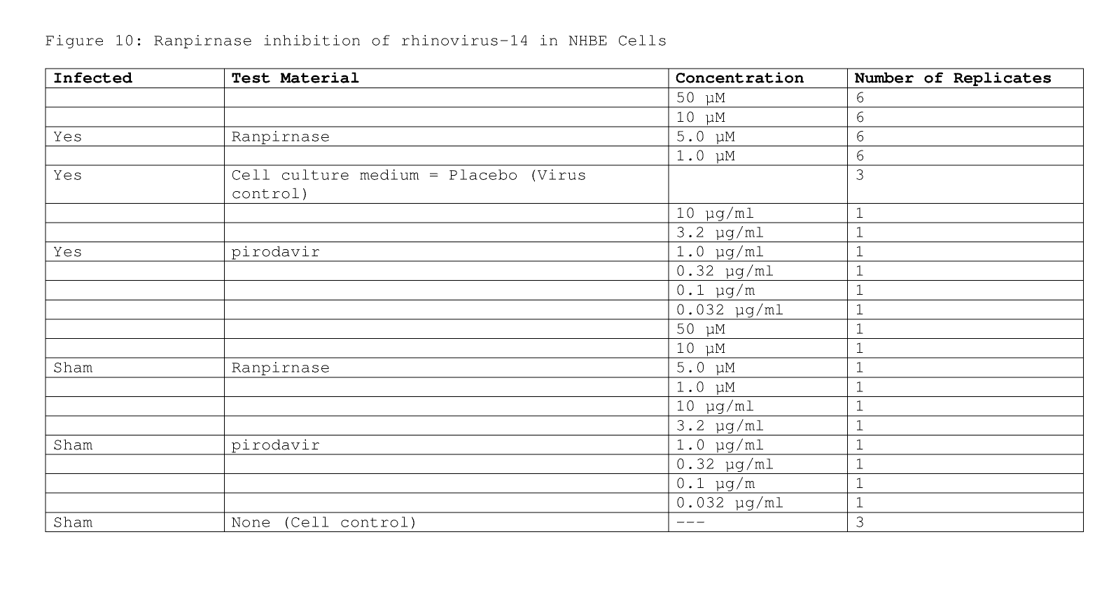

Differentiated NHBE cells were experimentally infected with RV-14 virus. After an adsorption period of 1 hour, the viral inoculum was removed and treatments applied (FIG. 10). Twenty-four hours post infection, treatments were replenished in the basal compartment of the tissue inserts. Four days post infection, supernatants were harvested and stored at −80° C. until determination of virus titers in HeLa-Ohio-1 cells (human cervical carcinoma cells from ATCC). Controls consisted of four groups:
Group 1—infected and placebo-treated cells (virus control);
Group 2—sham-infected and treated cells (toxicity controls);
Group 3—sham-infected and placebo-treated cells (cell control); and
Group 4—pirodavir as a positive control drug.

Toxicity controls were microscopically examined for possible changes in tissue and/or cell morphology at the end of the experiment.

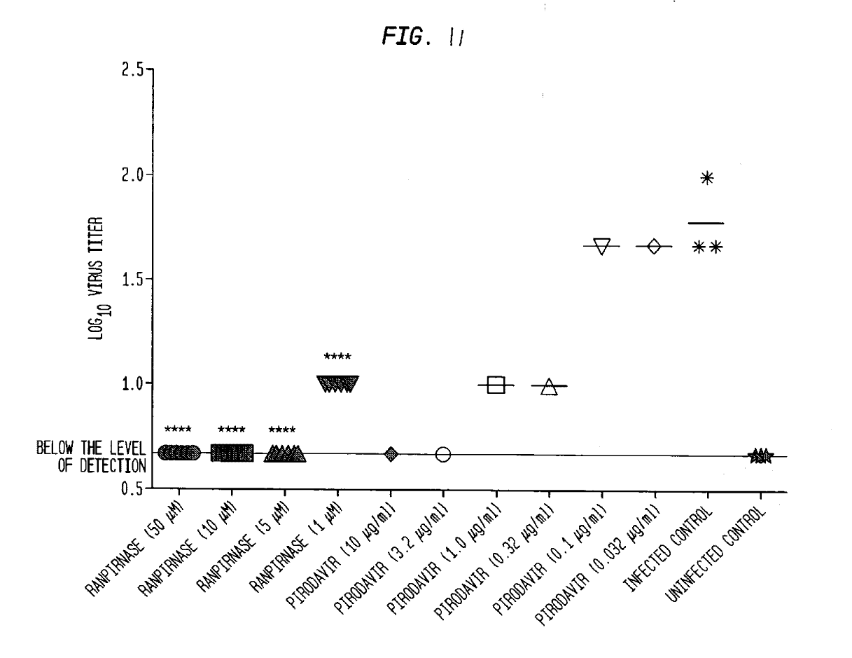

NHBE cells were inoculated by exposure of the apical side to RV-14 or cell culture medium (sham infection) as seen in FIG. 11. After 1 hour±10 min of incubation at 37° C. and 5% CO₂, the viral inoculum or cell culture medium was removed from the cells. The apical side of the cells was washed once with 500 μpre-warmed HEPES Buffered Saline Solution.

After inoculation, ranpirnase, pirodavir, or cell culture medium (placebo/cell control) was added to the apical side of the cells and in the basal medium compartment, and incubated with the cells for 1 hour. After 1-hour incubation, the drug-containing medium was removed from the apical and basal chambers. Culture medium alone (Placebo/Cell control) or with drug (test condition) was added to the bottom chamber, and cells were incubated for 4 days. Twenty-four hours post infection, cell culture medium with and without drug was replenished to the basal compartment.

Following infection and treatment, cells were maintained at the air-liquid interface, and cell culture supernatant was harvested 4 days post virus exposure. Virus released into the apical compartment of the NHBE cells was harvested by the addition and collection of 500 μl culture medium allowed to equilibrate for 30 min at 37° C. and 5% CO₂. The medium from the apical compartment divided into 2 aliquots, which were stored at −80° C. for future analysis of viral titers.

HeLa Ohio-1 cells were seeded in 96-well plates and grown overnight to achieve confluence, then washed twice with 100 μl infection medium (MEM/EBSS supplemented with 50 μl/ml gentamycin). Wells were filled with 100 μl infection medium. Apical washes from the NHBE cell cultures were diluted 10-fold in infection medium and 100 μl were transferred into respective wells of a 96-well microtiter plate. Each concentration of ranpirnase from the NHBE cells (6 NHBE cell wells/dose) was titered leading to six titers per concentration (each NHBE well treated as a replicate) to evaluate the virus yields from infected and infected, treated cells. Thus, each concentration of Ranpirnase was titered a total of six times. For the positive control, pirodavir, one well of NHBE cells only was assigned to each concentration. Thus, each concentration was titered only once. Three wells were assigned as untreated, infected controls. They were titered once, resulting in three replicate untreated, infected control titers. After 7 days of incubation at 37° C. and 5% CO₂, cells were microscopically examined and scored for virus-induced CPE. A well was scored positive if any trace of CPE (cell lysis) was observed as compared with the uninfected control. CCID50 was calculated by the Reed-Muench method and the inverse of that dilution represented the virus titer.

All ranpirnase treatments decreased virus titers relative to the titers of untreated infected controls except for the lowest dose (FIG. 11). The reduction in virus titer with 50, 10, 5 μM ranpirnase treatment represented an approximate 1.67 log 10 drop in virus titer for the (P<0.0001). For the 1 μM ranpirnase treatment, an ˜1 log reduction in virus titers was detected when compared to the virus titers detected from the untreated, infected controls wells (P<0.0001). Pirodavir inhibited virus replication as expected at 10 and 3.2 μg/ml with a somewhat dose responsive decrease in virus yields at subsequent lower dilutions of drug. Typically at 0.0032 μg/ml, pirodavir is also inactive against RV-14 in a HeLa Ohio-1 cell culture antiviral system as was seen in NHBE cells in this experiment.

No virus cytopathic effects were detected in uninfected, ranpirnase-treated or pirodavir-treated cells. Microscopy evaluations of ranpirnase-treated or pirodavir-treated NHBE cells revealed no toxicological phenomena.

Therefore, the results of the study showed that all doses of ranpirnase tested (50 μM, 1.0 μM, 5 μM and 1 μM) reduced RV-14 titers in a statistically significant manner, and that ranpirnase alone did not elicit cytopathic effects in NHBE cells. Such titer reduction, accompanied by absence of cytopathic effects on the host cells, is reasonably correlated with usefulness of ranpirnase in treating RV-14. Because ranpirnase was so effective at inhibiting RV-14 while not killing the host cells, this experiment further evidences the likelihood that systemically administered ranpirnase will be useful in treating a mammalian subject infected with a virus, and particularly a mammalian subject infected with RV-14. Furthermore, the above-disclosed experimental results in Zika virus, VEEV, CHIV, and EBOV indicate that it should be possible to use ranpirnase as a prophylactic to prevent RV-14 infection.

Generally, in view of the different viruses that respond to treatment using ranpirnase, a person of ordinary skill in this art would conclude that any route by which ranpirnase is systemically administered will be adequate to treat any particular virus (although one route may be more effective than another in any particular instance). Thus, enteral administration (including without limitation oral administration and rectal administration) and parenteral administration (including without limitation intravenous administration, intramuscular administration, and aerosol delivery) are appropriate methods for administration of ranpirnase.

A therapeutically effective dose of ranpirnase can be determined by the skilled person as a matter of routine experimentation. The therapeutically effective dosage of a pharmaceutical composition can be determined readily by the skilled artisan, for example, from animal studies. Also, human clinical studies can be performed to determine the preferred effective dose for humans by a skilled artisan. Such clinical studies are routine and well known in the art. The precise dose to be employed will also depend on the route of administration. Effective doses may be extrapolated from dose-response curves derived from in vitro or animal test systems.

The above-recited experimental results were carried out using ranpirnase. However, other ribonucleases that are highly homologous to ranpirnase have exhibited highly similar activities against other viruses. These other ribonucleases are identified in U.S. Pat. Nos. 5,728,805, 6,239,257, and U.S. Pat. No. 7,229,824. The RNase of SEQ ID NO:2 in U.S. Pat. No. 5,728,805 is herein referred to as the “'805 variant”, the RNase of SEQ ID NO:1 in U.S. Pat. No. 6,239,257 is herein referred to as “Amphinase 2”, and the RNase of SEQ ID NO:59 of U.S. Pat. No. 7,229,824 is herein referred to as “rAmphinase 2”. To a person of ordinary skill in this art, the similarities of homology and activity of these three other ribonucleases is strong evidence that these three other ribonucleases will have the same activity as ranpirnase has. Hence, although the above-disclosed experiments have not yet been repeated using the '805 variant, Amphinase 2, or rAmphinase 2, it is believed that the above data are fully applicable to these three ribonucleases and that these three ribonucleases will be active against Zika virus, MERS-CoV, CHIV, and RV in humans, and VEEV in equine species.

As demonstrated above, ranpirnase inhibits growth of Zika virus, MERS-CoV, VEEV, and CHIV, and RV-14 in various cell types. These five viruses are all categorized in Baltimore Classification Group IV. This activity, taken together with the above-disclosed activity that ranpirnase has demonstrated against a broad spectrum of viruses, is substantial evidence justifying the conclusion that systemically administered ranpirnase will be effective against viruses categorized in Baltimore Classification Group IV. And, based upon the similarities of homology and activity of the '805 variant, Amphinase 2, and rAmphinase 2 to the homology and activity of ranpirnase, these three other ribonucleases would be expected to have the same activity as ranpirnase against viruses classified in Baltimore Classification Group IV.

Although at least one preferred embodiment of the invention has been described above, this description is not limiting and is only exemplary. The scope of the invention is defined only by the claims, which follow:

1. A method of treating a viral infection in a mammalian patient, the viral infection being other than Dengue fever, yellow fever, and severe acute respiratory syndrome and being classified in Baltimore Classification Group IV, comprising systemically administering a therapeutically effective dose of ranpirnase to the patient.

2. A method of treating a viral infection in a mammalian patient, the viral infection being other than Dengue fever, yellow fever, and severe acute respiratory syndrome and being classified in Baltimore Classification Group IV, comprising systemically administering a therapeutically effective dose of Amphinase 2 to the patient.

3. A method of treating a viral infection in a mammalian patient, the viral infection being other than Dengue fever, yellow fever, and severe acute respiratory syndrome and being classified in Baltimore Classification Group IV, comprising systemically administering a therapeutically effective dose of rAmphinase 2 to the patient.

4. A method of treating a viral infection in a mammalian patient, the viral infection being other than Dengue fever, yellow fever, and severe acute respiratory syndrome and being classified in Baltimore Classification Group IV, comprising systemically administering a therapeutically effective dose of the '805 variant to the patient.

5. The method of claim 1, 2, 3, or 4, wherein the patient is a human being.

6. The method of claim 1, 2, 3, or 4 wherein the patient is an equine species.

7. A method of treating a Zika virus infection in a mammalian patient, comprising systemically administering a therapeutically effective dose of ranpirnase to the patient.

8. A method of treating a MERS-CoV infection in a mammalian patient, comprising systemically administering a therapeutically effective dose of ranpirnase to the patient.

9. A method of treating an infection in the rhinovirus family in a mammalian patient, comprising systemically administering a therapeutically effective dose of ranpirnase to the patient.

10. A method of treating a Venezuelan equine encephalitis infection in a mammalian patient, comprising systemically administering a therapeutically effective dose of ranpirnase to the patient.

11. A method of treating a Chikungunya infection in a mammalian patient, comprising systemically administering a therapeutically effective dose of ranpirnase to the patient.

12. The method of claim 7, 8, 9, or 11, wherein the patient is a human being.

13. The method of claim 10, wherein the patient is an equine species.

14. A method of treating a Zika virus infection in a mammalian patient, comprising systemically administering a therapeutically effective dose of Amphinase 2 to the patient.

15. A method of treating a MERS-CoV infection in a mammalian patient, comprising systemically administering a therapeutically effective dose of Amphinase 2 to the patient.

16. A method of treating an infection in the rhinovirus family in a mammalian patient, comprising systemically administering a therapeutically effective dose of Amphinase 2 to the patient.

17. A method of treating a Venezuelan equine encephalitis infection in a mammalian patient, comprising systemically administering a therapeutically effective dose of Amphinase 2 to the patient.

18. A method of treating a Chikungunya infection in a mammalian patient, comprising systemically administering a therapeutically effective dose of Amphinase 2 to the patient.

19. The method of claim 14, 15, 16, or 18, wherein the patient is a human being.

20. The method of claim 17, wherein the patient is an equine species.

21. A method of treating a Zika virus infection in a mammalian patient, comprising systemically administering a therapeutically effective dose of rAmphinase 2 to the patient.

22. A method of treating a MERS-CoV infection in a mammalian patient, comprising systemically administering a therapeutically effective dose of rAmphinase 2 to the patient.

23. A method of treating an infection in the rhinovirus family in a mammalian patient, comprising systemically administering a therapeutically effective dose of rAmphinase 2 to the patient.

24. A method of treating a Venezuelan equine encephalitis infection in a mammalian patient, comprising systemically administering a therapeutically effective dose of rAmphinase 2 to the patient.

25. A method of treating a Chikungunya infection in a mammalian patient, comprising systemically administering a therapeutically effective dose of rAmphinase 2 to the patient.

26. The method of claim 21, 22, 23, or 25 wherein the patient is as human being.

27. The method of claim 24, wherein the patient is an equine species.

28. A method of treating a Zika virus infection in a mammalian patient, comprising systemically administering a therapeutically effective dose of the '805 variant to the patient.

29. A method of treating a MERS-CoV infection in a mammalian patient, comprising systemically administering a therapeutically effective dose of the '805 variant to the patient.

30. A method of treating an infection in the adenoviridae family, or an infection in the rhinovirus family in a mammalian patient, comprising systemically administering a therapeutically effective dose of the '805 variant to the patient.

31. A method of treating a Venezuelan equine encephalitis infection in a mammalian patient, comprising systemically administering a therapeutically effective dose of the '805 variant to the patient.

32. A method of treating a Chikungunya infection in a mammalian patient, comprising systemically administering a therapeutically effective dose of the '805 variant to the patient.

33. The method of claim 28, 29, 30, or 32, wherein the patient is a human being.

34. The method of claim 31, wherein the patient is an equine species.

35. A method of prophylactically protecting a patient from:
a. Zika virus; or
b. MERS-CoV; or
c. Chikungunya; or
d. Venezuelan equine encephalitis; or
e. a virus in the rhinovirus family, comprising the step of administering a therapeutically effective dose of ranpirnase to the patient.

36. A method of prophylactically protecting a patient from:
a. Zika virus; or
b. MERS-CoV; or
c. Chikungunya; or
d. Venezuelan equine encephalitis; or
e. a virus in the rhinovirus family, comprising the step of administering a therapeutically effective dose of the '805 variant to the patient.

37. A method of prophylactically protecting a patient from:
a. Zika virus; or
b. MERS-CoV; or
c. Chikungunya; or
d. Venezuelan equine encephalitis; or
e. a virus in the rhinovirus family, comprising the step of administering a therapeutically effective dose of Amphinase 2 to the patient.

38. A method of prophylactically protecting a patient from:
a. Zika virus; or
b. Mers-CoV; or
c. Chikungunya; or
d. Venezuelan equine encephalitis; or
e. a virus in the rhinovirus family, comprising the step of administering a therapeutically effective dose of rAmphinase 2 to the patient.

39. A method of treating a patient for a viral infection caused by a virus in the Flaviviridae family, comprising systemically administering a therapeutically effective dose of ranpirnase to the patient.

40. A method of treating a patient for a viral infection caused by a virus in the Flaviviridae family, comprising systemically administering a therapeutically effective dose of the '805 variant to the patient.

41. A method of treating a patient for a viral infection caused by a virus in the Flaviviridae family, comprising systemically administering a therapeutically effective dose of Amphinase 2 to the patient.

42. A method of treating a patient for a viral infection caused by a virus in the Flaviviridae family, comprising systemically administering a therapeutically effective dose of rAmphinase 2 to the patient.
